(19) United States
(12) Patent Application Publication
(10) Pub. No.: US 2006/0110471 A1
(43) Pub. Date: May 25, 2006

(54) Essential oil blend for inhalation

(76) Inventor: Wendy S. Nichols, Kailua, HI (US)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 10/993,653

(22) Filed: Nov. 19, 2004

(51) Int. Cl. A61K 36/53; A61K 36/534; A61K 36/9068

(52) U.S. Cl. 424/745; 424/747; 424/756

(57) ABSTRACT

The present invention provides a novel blend of essential oils and herbs. A blend of the invention may be used to treat nausea. A blend of the invention may be used to provide a calming, soothing feeling. Optionally the novel blend of the invention is introduced into the body of a person by inhalation. In one aspect of the invention, the novel blend of essential oils and herbs includes Mentha piperita (peppermint), Mentha spicata (spearmint), Zingiber officinale (ginger), and Lavandula angustifolia (lavender), as well as the herbs Lavandula angustifolia (lavender), Ocimum basilicum (basil), Artemesia dracunculus (tarragon) and Mentha spicata (spearmint).

### STATEMENT OF FEDERALLY SPONSORED RESEARCH

Not applicable.

### BACKGROUND OF THE INVENTION

1. Field of the Invention

This invention relates to a novel essential oil and herb mixture for prevention and/or treatment of nausea, and a novel essential oil and herb mixture for providing a calming, soothing feeling, methods for treatment of nausea, methods for providing a calming, soothing feeling.

2. Description of the Background Art

Essential oils, for example, may be complex mixtures of naturally occurring compounds that exist in some plants' flowers, leaves, wood, bark, roots, or seeds. Active ingredients in essential oils are said to include, but are not limited to, hydrocarbons, including terpenes, esters, alcohols, aldehydes, ketones, and phenols. Essential oils and their natural ingredients may also be made synthetically, though many proponents of essential oils believe that synthetic essential oils lack some of the efficacy of natural essential oils.

Although Applicants of course do not wish to be bound by any particular theory, it has been speculated that plants developed the active ingredients in their essential oils as defenses against such things as insect attacks, herbivores, bacterial and fungal infestation. It has also been speculated that essential oils were developed to aid pollination, to help heal wounds inflicted on the plant, and to assist survival in difficult growth conditions.

Those skilled in the art of essential oils will recognize that oils may be extracted from plants using a variety of acceptable methods. Generally, methods used to extract oils for use in therapy are non-toxic, though toxic methods are also used if the toxicity can later be eliminated. Some non-toxic methods of extraction include distillation and cold-pressing. Particularly useful non-toxic methods of extraction preserve quality, purity, and wholesomeness of the extracted oils. Those skilled in the art will also recognize that there may be some minor variation in the composition of essential oils, based on growth, processing, and other possible variables; however, correctly processed oils will retain those components useful for producing an overall therapeutic effect.

Different molecular components of essential oils may be introduced to the body of a patient in a number of ways, including but not limited to topical application, internal application, oral ingestion, and inhalation. For instance, U.S. Pat. No. 6,280,751, to Fletcher, et al., incorporated by reference herein, reports use of essential oils in combination with aloe for topical application or consumption.

Although Applicants do not wish to be bound by theory, it is believed that inhalation is an ideal method for introduction of essential oils into the body because it is fast, safe, and uncomplicated. Following inhalation of an essential oil blend, molecules of the essential oil may travel up the nasal passage to the olfactory bulb, where chemical messengers are dispensed into the limbic system of the brain. From the limbic system, messages are forwarded to areas of the central nervous system that mediate emotional, hormonal, metabolic and stress responses throughout the body.

Nausea and vomiting are both unpleasant effects with a variety of possible causes and contributing factors. Nausea may be brought about, for instance, as an after-effect of surgery, as a result of excessive motion, or by other factors, including blood-borne substances and tactile sensations.

As reported in U.S. Pat. No. 6,030,631, to Langevin, incorporated by reference herein, many medical alternatives used to treat nausea are not entirely satisfactory for one or more of a number of reasons. For instance, frequently used anti-nausea and anti-emetic drugs such as benzamides, phenothiazines, and serotonin inhibitors may be expensive and may have the potential for side effects. These drugs may also require hospitalization during use, and may require the added expense and inconvenience of requiring administration by a healthcare professional.

It would be helpful to have a treatment for nausea that is fast, effective, inexpensive, natural and/or safe. It would also be helpful to have a method and composition for providing a calming, soothing feeling. It is an object of the instant invention to provide an essential oil and herb blend that satisfies one or more than one of the goals of efficacy, economy, naturalness, or safety. Of course, the breadth and scope of the invention as defined by the claims should not be limited by reference to any object of the invention.

### SUMMARY OF THE INVENTION

In one aspect the invention includes a composition for treatment of nausea comprising essential oils, wherein the essential oils comprise ginger, spearmint, lavender, and peppermint. A further aspect of the invention includes essential oils wherein the ratio of lavender to peppermint to ginger to spearmint is about 9:9:1:1. In another aspect of the invention, the lavender has a linolol content greater than about 20%.

In a yet further aspect of the invention, the essential oils are about 15% to about 80% lavender, about 15% to about 60% peppermint, about 5% to about 30% ginger, and about 5% to about 30% spearmint. In a further aspect of the invention, the essential oils are about 20% to about 60% lavender, about 20% to about 60% peppermint, about 5% to about 15% ginger, and about 5% to about 15% spearmint. In a yet still further aspect of the invention, the said essential oils are 41% lavender, 41% peppermint, 9% ginger, and 9% spearmint.

In an other aspect of the invention, the composition comprises at least one herb. In a further aspect, at least one herb is selected from the group consisting of Lavandula angustifolia (lavender), Mentha piperita (peppermint), Ocimum basilicum (basil), Artemesua dracunculus (tarragon), Mentha spicata (spearmint), Lavandula officinalis (lavender), and Lavandula intermedia (lavender). In a further aspect of the invention, the composition comprises the herbs Lavandula angustifolia (lavender), Mentha piperita (peppermint), Ocimum basilicum (basil), Artemesua dracunculus (tarragon), and Mentha spicata (spearmint). In another aspect, the composition further comprises the herbs Lavandula angustifolia (lavender), Mentha piperita (peppermint), Ocimum basilicum (basil), and Artemesua dracunculus (tarragon). Herbs of the invention may be provided, for example, in an emulsion, a suspension, or in ground form.

A further aspect of the invention includes wherein one or more of the essential oils discussed above is replaced with or supplemented with at least one of the essential oils Elettaria cardamomum (cardomom), Lavandula intermedia CT Super (Lavender), Eugenia caryophyllata (clove), Foeniculum vulgare (fennel), Anethum graveolens (aniseed), Citrus aurantium (petitgrain), Citrus bergamia (bergamot), Citrus reticulate (mandarin), Coriander sativum (coriander), Eucalyptus radiate (black peppermint), Foeniculum vulgare (sweet fennel), Hyssopus officinalis (hyssop), Juniperus communis juniper berry), Melaleuca alternifolia (tea tree), Myristica fragrans (nutmeg), Origanum maforana (sweet marjoram), Rosmarunus officinalis (rosemary), Salvia officinalis (sage), Thymus satureioides (Moroccan or Borneol thyme), Thymus vulgaris (thyme), and Lavandula officinalis (lavender).

A further aspect of the invention includes a method for treating nausea comprising selecting a patient who is nauseous, providing a composition of the invention, and administering the composition to the patient. The administration may be performed by the patient.

An aspect of the invention also includes an article of manufacture, comprising, packaged together, a composition of the invention, a delivery system for inhalation of an aroma created by the composition, and optionally instructions.

In a further aspect of the invention, the composition further comprises a carrier. In another aspect the carrier is selected from a wax medium, a gel medium and vinyl acetate beads.

In a further aspect, the invention is directed to a blend of herbs and essential oils. The novel blends of the invention may be used to treat, for example, nausea, for instance by inhalation of an aroma arising out of a novel blend of the invention.

In one aspect the invention includes a composition for providing a calming, soothing feeling, comprising essential oils, wherein the essential oils comprise ginger, spearmint, lavender, and peppermint. A further aspect of the invention includes essential oils wherein the ratio of lavender to peppermint to ginger to spearmint is about 9:9:1:1. In another aspect of the invention, the lavender has a linolol content greater than about 20%.

In a yet further aspect of the invention, the essential oils are about 15% to about 80% lavender, about 15% to about 60% peppermint, about 5% to about 30% ginger, and about 5% to about 30% spearmint. In a further aspect of the invention, the essential oils are about 20% to about 60% lavender, about 20% to about 60% peppermint, about 5% to about 15% ginger, and about 5% to about 15% spearmint. In a yet still further aspect of the invention, the said essential oils are 41% lavender, 41% peppermint, 9% ginger, and 9% spearmint.

In an other aspect of the invention, the composition comprises at least one herb. In a further aspect, at least one herb is selected from the group consisting of Lavandula angustifolia (lavender), Mentha piperita (peppermint), Ocimum basilicum (basil), Artemesua dracunculus (tarragon), Mentha spicata (spearmint), Lavandula officinalis (lavender), and Lavandula intermedia (lavender). In a further aspect of the invention, the composition comprises the herbs Lavandula angustifolia (lavender), Mentha piperita (peppermint), Ocimum basilicum (basil), Artemesua dracunculus (tarragon), and Mentha spicata (spearmint). In another aspect, the composition further comprises the herbs Lavandula angustifolia (lavender), Mentha piperita (peppermint), Ocimum basilicum (basil), and Artemesua dracunculus (tarragon). Herbs of the invention may be provided, for example, in an emulsion, a suspension, or in ground form.

A further aspect of the invention includes wherein one or more of the essential oils discussed above is replaced with or supplemented with at least one of the essential oils Elettaria cardamomum (cardomom), Lavandula intermedia CT Super (Lavender), Eugenia caryophyllata (clove), Foeniculum vulgare (fennel), Anethum graveolens (aniseed), Citrus aurantium (petitgrain), Citrus bergamia (bergamot), Citrus reticulate (mandarin), Coriander sativum (coriander), Eucalyptus radiate (black peppermint), Foeniculum vulgare (sweet fennel), Hyssopus officinalis (hyssop), Juniperus communis (juniper berry), Melaleuca alternifolia (tea tree), Myristica fragrans (nutmeg), Origanum maforana (sweet marjoram), Rosmarunus officinalis (rosemary), Salvia officinalis (sage), Thymus satureioides (Moroccan or Borneol thyme), Thymus vulgaris (thyme), and Lavandula officinalis (lavender).

A further aspect of the invention includes a method for providing a calming, soothing feeling, comprising selecting aperson who desires a calming, soothing feeling, providing a composition of the invention, and administering the composition to the person. The administration may be performed by the person.

An aspect of the invention also includes an article of manufacture, comprising, packaged together, a composition of the invention for providing a calming, soothing feeling, a delivery system for inhalation of an aroma created by the composition, and optionally instructions.

In a further aspect of the invention, the composition for providing a calming, soothing feeling further comprises a carrier. In another aspect the carrier is selected from a wax medium, a gel medium and vinyl acetate beads.

In a further aspect, the invention is directed to a blend of herbs and essential oils. The novel blends of the invention may be used, for example, to provide a calming, soothing feeling, for instance by inhalation of an aroma arising out of a novel blend of the invention.

### DETAILED DESCRIPTION OF THE INVENTION

In one aspect, the invention provides a novel composition that may be made from a variety of essential oils. For example, one composition of the invention includes the essential oils Mentha piperita (peppermint), Mentha spicata (spearmint), Zingiber officinale (ginger), and Lavandula angustifolia (lavender), as well as the herbs Lavandula angustifolia (lavender), Mentha piperita (peppermint), Ocimum basilicum (basil), and Artemesia dracunculus (tarragon). In other aspects of the invention, various essential oils and herbs listed above are optional ingredients.

Essential oils of the invention may be included in varying amounts. One aspect of the invention includes an essential oil content of about 41% lavender, about 41% peppermint, about 9% ginger, and about 9% spearmint. In a further aspect the invention includes about 30% to about 50% lavender, about 30% to about 50% peppermint, about 5% to about 15% ginger, and about 5% to about 15% spearmint. In a yet further aspect the invention includes about 20% to about 60% lavender, about 20% to about 60% peppermint, about 5% to about 15% ginger, and about 5% to about 15% spearmint. In a yet still further aspect the invention includes about 15% to about 80% lavender, about 15% to about 60% peppermint, about 5% to about 30% ginger, and about 5% to about 30% spearmint. Those skilled in the art will recognize that varying the amount of ingredients may vary the effects of the composition.

Herbs may also be included in a variety of amounts in compositions of the invention. For instance, in one aspect a composition of the invention may include about 25% lavender, about 25% peppermint, about 25% basil, and about 25% tarragon. In a further aspect of the invention, a composition may include between about 0% to about 50% lavender, about 0% to about 50% peppermint, about 0% to about 50% basil, and about 0% to about 50% tarragon.

Of the components of an exemplary composition of the invention, peppermint is said to have efficacy in treating postoperative nausea and vomiting, chemotherapy induced nausea, and colon spasms. Although applicants do not wish to be bound by theory, peppermint is believed to exert an antispasmodic influence on the gastric lining and colon of a patient through alcohol compounds such as menthone and menthol. Peppermint is also believed to help provide a calming, soothing feeling to a person. Spearmint was selected for use based on similar anti-emetic benefits, as well as for the possibility that it is effective in reducing nausea and/or emesis for longer periods than peppermint alone. Spearmint may also provide a user of a composition of the invention with a feeling that they are taking deeper breaths.

Although essential oils of the composition are listed in the specification under scientific names, it is also contemplated that different species recognized by those skilled in the art to yield similar or identical essential oils may be substituted.

Ginger may also be included in an exemplary composition of the invention. Ginger includes zingiberene, which is believed to play some role in the anti-inflammatory and anti-emetic properties of ginger. Ginger has a pungent scent, but this scent may be modified or overcome by mixture with other essential oils and herbs. Lavender is said to have sedative effects in both animals and humans, and may have some effect on nausea when inhaled independently of the other ingredients of the novel composition.

The herbs included with the exemplary composition listed above may have a number of functions. For example, they may alter the scent of the composition in a favorable manner (or, if desired, an unfavorable manner), they may alter the texture of the composition's aroma, or they may provide other beneficial effects in addition to those of the essential oils of the composition.

A number of essential oils, in addition to those listed above, may be used in the invention. Ideally, though not necessarily, essential oils used in the invention may have some anti-nausea or anti-emetic properties. Particularly useful are lavender oils that have a high linolol content, including those that have a linolol content of greater than about 20%, those that have a linolol content of greater than about 30%, and those that have a linolol content greater than about 40%. Combinations of oils with varying linolol contents may also be used. Other essential oils may be used in addition to, or in lieu of, those listed in the exemplary aspect of the invention given above. A non-limiting list of essential oils that may be used in varying combinations in the invention are set forth in Table 1, below. These essential oils may be used in the composition in lieu of, or in addition to, the oils previously discussed. Essential oils containing borneol and myrcene may provide relief from nausea due to their anticholinergic activity. Essential oils with significant percentages of these molecules are included in the list below.

TABLE 1

| | |
|---|---|
| Elettaria cardamomum | Cardomom |
| Lavandula Intermedia CT Super | Lavender |
| Eugenia caryophyllata | Clove |
| Foeniculum vulgare | Fennel |
| Anethum Graveolens | Aniseed |
| Citrus aurantium | Petitgrain |
| Citrus bergamia | Bergamot |
| Citrus Reticulata | Mandarin |
| Coriander Sativum | Coriander |
| Eucalyptus radiate | Black Peppermint |
| Foeniculum vulgare | Sweet Fennel |
| Hyssopus Officinalis | Hyssop |
| Juniperus communis | Juniper Berry |
| Melaleuca alternifolia | Tea Tree |
| Myristica fragrans | Nutmeg |
| Origanum maforana | Sweet Marjoram |
| Rosmarunus officinalis | Rosemary |
| Salvia officinalis | Sage |
| Thymus satureioides | Moroccan or Borneol Thyme |
| Thymus vulgaris | Thyme |
| Lavandula officinalis | Lavender |

A number of herbs, in addition to those listed above, may also be used in the invention. In some aspects, compositions of the invention may include no herbs at all. In some aspects of the invention, a composition includes herbs and oils in an approximate ratio of about 2 grams of herbs to about 2.2 ml of oils. Herbs may be used in addition to, or in lieu of, those listed in the exemplary aspect of the invention given above. A non-limiting list of herbs that may be used in varying combinations in the invention are set forth in Table 2, below.

TABLE 2

| | |
|---|---|
| Ocimum basilicum | Basil |
| Artemisia dracunculus | Tarragon |
| Mentha spicata | Spearmint |
| Mentha piperita | Peppermint |
| Lavandula angustifolia | Lavender |
| Lavandula officinalis | Lavender |
| Lavandula intermedia | Lavender |

Compositions of the invention may also include optional additives. Additives may include, for example, but are not limited to, natural and/or synthetic preservatives Other additives may include oils that are used to dilute essential oils used in the invention, such as jojoba, grape seed, almond oil, or herbs or other substances used to dilute the herbs that may be used in compositions of the invention. Some inert substances may be included in the invention as well. Such inert substances may be included to aid delivery of the aroma of a composition of the invention. Such inert substances may be substrates, for example wax and/or synthetic beads, which may be vinyl acetate beads such as Exvax 256, a product of DuPont. The invention also contemplates, though it does not prefer, inclusion of impurities in the essential oils and herbs, so long as such impurities do not change the essential nature of the composition.

Substances may also be included in a composition of the invention to enable the composition to be provided as an emulsion, a suspension, a gel, or other convenient or desired form depending on the delivery system to be used. Potential substances for inclusion may be found in the International Cosmetic Ingredient Dictionary and Handbook, 10th Edition, available from the Cosmetic, Toiletry, and Fragrance Association, which is incorporated by reference herein.

Compositions of the invention are suitable for use with a number of delivery systems. Such delivery systems may be designed, for example, for a person seeking treatment to inhale the aroma of the composition without assistance. They may be designed for administration by another. They may also be automated, for instance by use of a ventilation system that adds the aroma of a composition to the air. Delivery systems may be portable or fixed. Delivery systems may be disposable after one use or multiple uses, or they may be designed for replacement of a composition following loss of efficacy of the composition. A delivery system may also be designed to keep components of the composition separate until use is imminent, at which time a user or administrator may mix the ingredients and prepare the delivery system.

Delivery systems for use with the invention may contain components in addition to those in the composition, many of which may function to retain the composition in the delivery system. These components may include beads, wax, cotton, shaped cotton, or small permeable or semi-permeable bags. For instance, a delivery system may include at least one sachet containing herbs of a composition of the invention. The sachet or sachets may have been contacted and/or saturated with essential oils of a composition of the invention. In some aspects of the invention, a sachet is placed between two cotton pads, or under a cotton pad, or within a cotton pad that has a pocket form. A cotton pad may be placed within the sachet. The delivery system may then contain an effective amount of essential oils and herbs of a composition of the invention.

In another aspect, a composition of the invention can be packaged as part of a kit, with a container that holds or may hold an effective amount of the composition. The kit may include instructions. The kit may include a number of containers that separate oils and herbs of the composition until use. The kit may include all or some of the ingredients in a premixed fashion.

In a further aspect, the invention includes a method for treating nausea by inhalation of a composition of the invention, where the composition includes at least one essential oil recited herein. The method includes obtaining a composition of the invention, then administering an effective amount of the composition to a patient by inhalation of the aroma of the composition. Inhalation may be repeated, and may be shallow or deep. The composition may be self-administered by a patient in need of treatment. The composition may be administered by a third party to a patient in need of treatment, or a third party may assist a patient.

In a yet still further aspect, the invention includes a method for providing a calming, soothing feeling to a person by inhalation of a composition of the invention, where the composition includes at least one essential oil recited herein. The method includes obtaining a calming, soothing composition of the invention, then administering an effective amount of the composition to a person by inhalation of the aroma of the composition. Inhalation may be repeated, and may be shallow or deep. The composition may be self-administered by a person in need of a calming, soothing feeling. The composition may be administered by a third party to a person who desires a calming, soothing feeling, or a third party may assist such a person.

Treatment of nausea may include complete or partial alleviation of nausea as determined by questioning a patient or by other relevant methods that may be recognized by those skilled in the art. By inhalation of an aroma from a composition of the invention, nausea may be decreased, for example, by at least about 10%, by at least about 20%, by at least about 30%, by at least about 40%, by at least about 50%, by at least about 60%, by at least about 70%, by at least about 80%, by at least about 90%, or by about 100%.

### EXAMPLES

Examples 1 and 2 describe methods of making an essential oil and herb mixture of the invention in which the delivery system for inhalation includes a tin.

### Example 1

Approximately 1 g of each of the herbs Ocimum basilicum (basil), Artemisia dracunculus (tarragon), Mentha spicata (spearmint), Mentha piperita (peppermint), and Lavandula angustifolia (lavender) was placed inside a heat-sealable hydrogen peroxide bleached tea bag, creating an herb sachet. The open edge of the tea bag was sealed using a flat iron heated to approximately 300° F. A round absorbent cotton pad was placed in the bottom of a coated 2.25 inch diameter round, 1.0 inch deep tin container, and the herb sachet was placed on that cotton pad.

Each essential oil was drawn into a separate glass pharmaceutical dropper, and a selected amount of each oil was placed into a common glass container. The essential oils used included Lavandula angustifolia (lavender), Mentha piperita (peppermint), Zingiber officinale (ginger), and Mentha spicata (spearmint) in amounts of 18 drops lavender, 18 drops peppermint, 4 drops ginger, and 4 drops spearmint. One milliliter is about 20 drops.

The essential oil mixture was shaken vigorously and stirred to combine the essential oils, and a clean pharmaceutical dropper was used to draw the essential oil mixture. Forty-four drops of the essential oil mixture were used to saturate the sachet and the underlying cotton pad. A second cotton pad was placed over the saturated sachet and first cotton pad, and a round wire cover was secured by friction in the tin container, to prevent the pads and sachet from escaping. A removable, secure cover was placed on the tin.

When the cover of the tin was removed, the resulting aroma was effective to reduce nausea. The resulting aroma was also effective to provide a calming, soothing feeling. The tin delivery system was easily portable.

### Example 2

Construction of the composition and assembly of the delivery system in Example 2 were the same as in Example 1. The only exception was the addition of the herbs, which were included in amounts of 0.5 g each. The resulting aroma was less intense than that emanating from the composition of Example 1.

Patents, patent applications, publications, scientific articles, books, and other documents and materials referenced or mentioned herein are indicative of the levels of skill of those skilled in the art to which the inventions pertain. Each such referenced document and material is hereby incorporated by reference to the same extent as if it had been incorporated by reference in its entirety individually or set forth or reprinted herein in its entirety. Additionally, all claims in this application, and all priority applications, including but not limited to original claims, are hereby incorporated in their entirety into, and form a part of, the written description of the invention.

The inventions have been described broadly and generically herein. Each of the narrower species and subgeneric groupings falling within the generic disclosure also form part of these inventions. This includes the generic description of each invention which hereby include, including any claims thereto, a proviso or negative limitation removing or optionally allowing the removal of any subject matter from the genus, regardless of whether or not the excised materials or options were specifically recited or identified in haec verba herein, and all such variations form a part of the original written description of the inventions. In addition, where features or aspects of an invention are described in terms of a Markush group, the invention shall be understood thereby to be described in terms of each and every, and any, individual member or subgroup of members of the Markush group.

The methods and processes illustratively described herein may be suitably practiced in differing orders of steps. They are not necessarily restricted to the orders of steps indicated herein or in the claims. The compositions described and claimed herein may comprise, consist of, or consist essentially of any of the recited ingredients in any combination.

As used herein and in the appended claims, the singular forms “a,” “an,” and “the” include plural reference unless the context clearly dictates otherwise.

Under no circumstances may the patent be interpreted to be limited to the specific examples or aspects or methods specifically disclosed herein. Under no circumstances may the patent be interpreted to be limited by any statement made by any Examiner or any other official or employee of the Patent and Trademark Office unless such statement was specifically and without qualification or reservation expressly adopted by Applicants in a responsive writing specifically relating to the application that led to this patent prior to its issuance.

The terms and expressions employed herein have been used as terms of description and not of limitation, and there is no intention in the use of such terms and expressions, or any portions thereof, to exclude any equivalents now know or later developed, whether or not such equivalents are set forth or shown or described herein or whether or not such equivalents are viewed as predictable, but it is recognized that various modifications are within the scope of the invention claimed, whether or not those claims issued with or without alteration or amendment for any reason. Thus, it shall be understood that, although the present invention has been specifically disclosed by preferred embodiments and optional features, modifications and variations of the inventions embodied therein or herein disclosed can be resorted to by those skilled in the art, and such modifications and variations are considered to be within the scope of the inventions disclosed and claimed herein.

Specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. Where examples are given, the description shall not be construed to include but not to be limited to only those examples. It will be readily apparent to one skilled in the art that varying substitutions and modifications may be made to the invention disclosed herein without departing from the scope and spirit of the invention, and from the description of the inventions, including those illustratively set forth herein, it is manifest that various modifications and equivalents can be used to implement the concepts of the present invention without departing from its scope. A person of ordinary skill in the art will recognize that changes can be made in form and detail without departing from the spirit and the scope of the invention. The described embodiments are to be considered in all respects as illustrative and not restrictive. Thus, for example, additional embodiments are within the scope of the invention and within the following claims.

What is claimed is:

1. A composition for the treatment of nausea comprising essential oils, wherein said essential oils comprise ginger, spearmint, lavender, and, peppermint.

2. The composition of claim 1, wherein the ratio of lavender to peppermint to ginger to spearmint is about 9:9:1:1.

3. The composition of claim 1, wherein said essential oils comprise about 15% to about 80% lavender, about 15% to about 60% peppermint, about 5% to about 30% ginger, and about 5% to about 30% spearmint.

4. The composition of claim 3, wherein said essential oils comprise about 20% to about 60% lavender, about 20% to about 60% peppermint, about 5% to about 15% ginger, and about 5% to about 15% spearmint.

5. The composition of claim 4, wherein said essential oils comprise 41% lavender, 41% peppermint, 9% ginger, and 9% spearmint.

6. The composition of claim 1, further comprising at least one herb.

7. The composition of claim 6, wherein said at least one herb is selected from the group consisting of Lavandula angustifolia (lavender), Mentha piperita (peppermint), Ocimum basilicum (basil), Artemesua dracunculus (tarragon), Mentha spicata (spearmint), Lavandula officinalis (lavender), and Lavandula intermedia (lavender).

8. The composition of claim 1, further comprising the herbs Lavandula angustifolia (lavender), Mentha piperita (peppermint), Ocimum basilicum (basil), Artemesua dracunculus (tarragon), and Mentha spicata (spearmint).

9. The composition of claim 1, said composition further comprising Lavandula angustifolia (lavender), Mentha piperita (peppermint), Ocimum basilicum (basil), and Artemesua dracunculus (tarragon).

10. The composition of claim 9, wherein said herbs are provided in a suspension, emulsion, or as dry ground herbs.

11. The composition of claim 1, wherein at least one of said ginger and said lavender is replaced by an essential oil selected from the group consisting of Elettaria cardamomum (cardomom), Lavandula intermedia CT Super (Lavender), Eugenia caryophyllata (clove), Foeniculum vulgare (fennel), Anethum graveolens (aniseed), Citrus aurantium (petitgrain), Citrus bergamia (bergamot), Citrus reticulate (mandarin), Coriander sativum (coriander), Eucalyptus radiate (black peppermint), Foeniculum vulgare (sweet fennel), Hyssopus officinalis (hyssop), Juniperus communis (juniper berry), Melaleuca alternifolia (tea tree), Myristica fragrans (nutmeg), Origanum maforana (sweet marjoram), Rosmarunus officinalis (rosemary), Salvia officinalis (sage), Thymus satureioides (Moroccan or Borneol thyme), Thymus vulgaris (thyme), and Lavandula officinalis (lavender).

12. The composition of claim 1, wherein said lavender has a linolol content greater than about 20%.

13. A method for treating nausea, comprising:
(a) selecting a patient who is nauseous;
(b) providing an effective amount of the aroma of the composition of claim 1; and
(c) administering said aroma to said patient by inhalation.

14. The method of claim 13, wherein said administration is performed by said patient.

15. An article of manufacture, comprising, packaged together:
(a) the composition of claim 1;
(b) a delivery system for inhalation of an aroma, wherein said aroma is created by said composition; and
(c) optionally, instructions.

16. The article of manufacture of claim 15, wherein said composition further comprises the herbs Lavandula angustifolia (lavender), Mentha piperita (peppermint), Ocimum basilicum (basil), Artemesua dracunculus (tarragon), and Mentha spicata (spearmint).

17. The composition of claim 1, further comprising a carrier.

18. The composition of claim 17, wherein said carrier is selected from the group consisting of wax medium, gel medium, and vinyl acetate beads.

19. A composition for the treatment of nausea, consisting essentially of the essential oils lavender, peppermint, ginger, and spearmint, and the herbs lavender, spearmint, basil, and tarragon.

20. A method for treating nausea, comprising:
(a) selecting a patient who is nauseous;
(b) providing an effective amount of the aroma of a composition including at least two essential oils selected from the group consisting of Mentha piperita (peppermint), Mentha spicata (spearmint), Zingiber officinale (ginger), Lavandula angustifolia (lavender), Elettaria cardamomum (cardomom), Lavandula intermedia CT Super (Lavender), Eugenia caryophyllata (clove), Foeniculum vulgare (fennel), Anethum graveolens (aniseed), Citrus aurantium (petitgrain), Citrus bergamia (bergamot), Citrus reticulate (mandarin), Coriander sativum (coriander), Eucalyptus radiate (black peppermint), Foeniculum vulgare (sweet fennel), Hyssopus officinalis (hyssop), Juniperus communis (juniper berry), Melaleuca alternifolia (tea tree), Myristica fragrans (nutmeg), Origanum maforana (sweet marjoram), Rosmarunus officinalis (rosemary), Salvia officinalis (sage), Thymus satureioides (Moroccan or Borneol thyme), Thymus vulgaris (thyme), and Lavandula officinalis (lavender); and
(c) administering said aroma to said patient by inhalation.

21. A composition for providing a calming, soothing feeling, comprising essential oils, wherein said essential oils comprise ginger, spearmint, lavender, and, peppermint.

22. The composition of claim 21, wherein the ratio of lavender to peppermint to ginger to spearmint is about 9:9:1:1.

23. The composition of claim 21, wherein said essential oils comprise about 15% to about 80% lavender, about 15% to about 60% peppermint, about 5% to about 30% ginger, and about 5% to about 30% spearmint.

24. The composition of claim 23, wherein said essential oils comprise about 20% to about 60% lavender, about 20% to about 60% peppermint, about 5% to about 15% ginger, and about 5% to about 15% spearmint.

25. The composition of claim 24, wherein said essential oils comprise 41% lavender, 41% peppermint, 9% ginger, and 9% spearmint.

26. The composition of claim 21, further comprising at least one herb.

27. The composition of claim 26, wherein said at least one herb is selected from the group consisting of Lavandula angustifolia (lavender), Mentha piperita (peppermint), Ocimum basilicum (basil), Artemesua dracunculus (tarragon), Mentha spicata (spearmint), Lavandula officinalis (lavender), and Lavandula intermedia (lavender).

28. The composition of claim 21, further comprising the herbs Lavandula angustifolia (lavender), Mentha piperita (peppermint), Ocimum basilicum (basil), Artemesua dracunculus (tarragon), and Mentha spicata (spearmint).

29. The composition of claim 21, said composition further comprising Lavandula angustifolia (lavender), Mentha piperita (peppermint), Ocimum basilicum (basil), and Artemesua dracunculus (tarragon).

30. The composition of claim 29, wherein said herbs are provided in a suspension, emulsion, or as dry ground herbs.

31. The composition of claim 21, wherein at least one of said ginger and said lavender is replaced by an essential oil selected from the group consisting of Elettaria cardamomum (cardomom), Lavandula intermedia CT Super (Lavender), Eugenia caryophyllata (clove), Foeniculum vulgare (fennel), Anethum graveolens (aniseed), Citrus aurantium (petitgrain), Citrus bergamia (bergamot), Citrus reticulate (mandarin), Coriander sativum (coriander), Eucalyptus radiate (black peppermint), Foeniculum vulgare (sweet fennel), Hyssopus officinalis (hyssop), Juniperus communis (juniper berry), Melaleuca alternifolia (tea tree), Myristica fragrans (nutmeg), Origanum maforana (sweet marjoram), Rosmarunus officinalis (rosemary), Salvia officinalis (sage), Thymus satureioides (Moroccan or Borneol thyme), Thymus vulgaris (thyme), and Lavandula officinalis (lavender).

32. The composition of claim 21, wherein said lavender has a linolol content greater than about 20%.

33. A method for providing a calming, soothing feeling, comprising:
(a) selecting a person who desires a calming, soothing feeling;
(b) providing an effective amount of the aroma of the composition of claim 21; and
(c) administering said aroma to said person by inhalation.

34. The method of claim 33, wherein said administration is performed by said person.

35. An article of manufacture, comprising, packaged together:
(a) the composition of claim 21;
(b) a delivery system for inhalation of an aroma, wherein said aroma is created by said composition; and
(c) optionally, instructions.

36. The article of manufacture of claim 35, wherein said composition further comprises the herbs Lavandula angustifolia (lavender), Mentha piperita (peppermint), Ocimum basilicum (basil), Artemesua dracunculus (tarragon), and Mentha spicata (spearmint).

37. The composition of claim 21, further comprising a carrier.

38. The composition of claim 37, wherein said carrier is selected from the group consisting of wax medium, gel medium, and vinyl acetate beads.

39. A composition for providing a calming, soothing feeling, consisting essentially of the essential oils lavender, peppermint, ginger, and spearmint, and the herbs lavender, spearmint, basil, and tarragon.

40. A method for providing a calming, soothing feeling, comprising:
(a) selecting a person who desires a calming, soothing feeling;
(b) providing an effective amount of the aroma of a composition including at least two essential oils selected from the group consisting of Mentha piperita (peppermint), Mentha spicata (spearmint), Zingiber officinale (ginger), Lavandula angustifolia (lavender), Elettaria cardamomum (cardomom), Lavandula intermedia CT Super (Lavender), Eugenia caryophyllata (clove), Foeniculum vulgare (fennel), Anethum graveolens (aniseed), Citrus aurantium (petitgrain), Citrus bergamia (bergamot), Citrus reticulate (mandarin), Coriander sativum (coriander), Eucalyptus radiate (black peppermint), Foeniculum vulgare (sweet fennel), Hyssopus officinalis (hyssop), Juniperus communis (juniper berry), Melaleuca alternifolia (tea tree), Myristica fragrans (nutmeg), Origanum maforana (sweet marjoram), Rosmarunus officinalis (rosemary), Salvia officinalis (sage), Thymus satureioides (Moroccan or Borneol thyme), Thymus vulgaris (thyme), and Lavandula officinalis (lavender); and
(c) administering said aroma to said person by inhalation.
